(19) United States
(12) Patent Application Publication
(10) Pub. No.: US 2007/0122460 A1
(43) Pub. Date: May 31, 2007

(54) Hot-flash wipes and clothing

(76) Inventor: Yvonne Daily, Chicago, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(21) Appl. No.: 11/602,853

(22) Filed: Nov. 20, 2006

Related U.S. Application Data
(60) Provisional application No. 60/740,137, filed on Nov. 28, 2005.

(51) Int. Cl. A61K 9/70; A61K 36/534; A61K 36/28; B32B 27/04

(52) U.S. Cl. 424/443; 424/747; 424/764; 442/123

(57) ABSTRACT

Disclosed are cooling clothing and wipes to alleviate some of the symptoms of menopause, especially hot flashes and night sweats. A wipe is made of a wicking material that does not disintegrate or leave a residue as do current tissues. The wipe carries a cooling agent, such as menthol. When drawn across the skin, the wipe draws perspiration away from the skin while providing a cooling effect. The cooling agent is compatible with make-up. A soothing agent, such as black cohosh, can be added to relax the woman which in turn decreases the severity of the hot flash. Clothing items are made with characteristics similar to those of the wipes. Worn at night, pajamas made according to the present invention relieve menopausal symptoms and allow the woman to sleep with less difficulty. Worn during the day, such clothing relieves such symptoms without drawing attention to the wearer.

### CROSS-REFERENCE TO RELATED APPLICATION

This application claims priority to U.S. Provisional Patent Application 60/740,137, “Hot-Flash Wipes and Clothing,” which was filed on Nov. 28, 2005, and which is incorporated herein by reference in its entirety.

### FIELD OF THE INVENTION

The present invention relates generally to clothing and to wipes and, more particularly, to clothing and to wipes for relieving symptoms of menopause.

### BACKGROUND OF THE INVENTION

During menopause, levels of a woman's ovarian hormones, such as estrogen and progesterone, fluctuate. These fluctuations are believed to cause, at least in part, the well known symptoms of menopause which include hot flashes, night sweats, and difficulty in sleeping.

During a hot flash, the woman experiences a sensation of intense heat, probably as a result of her brain's thermoregulatory center acting irregularly under the influence of decreasing estrogen levels. To compensate for the perceived heat, the woman often sweats and flushes and then shivers. The timing of hot flashes is unpredictable, but they may be triggered by stress, high ambient temperatures, alcohol, and caffeine. A “night sweat” is a hot flash that occurs while the woman is sleeping. The resulting sweating and discomfort can make continued sleep difficult.

There is some evidence that various vitamins and herbs may provide some relief.

### BRIEF SUMMARY OF THE INVENTION

In view of the foregoing, the present invention provides cooling clothing and wipes to alleviate some of the symptoms of menopause, especially hot flashes and night sweats.

A wipe is made of a wicking material that does not disintegrate or leave a residue as do current tissues. Examples of appropriate material include woven cloth, non-woven cloth, polyester, and nylon. The wipe carries a cooling agent, such as menthol or eucalyptus oil. When drawn across the skin, the wipe draws perspiration away from the skin while providing a cooling effect. The cooling agent is compatible with make-up. The wipe may be disposable or reusable.

In some embodiments, the wipe carries other compounds to, for example, remove excess skin oil. A soothing agent, such as black cohosh, vitex, or dong quai, can be added to relax the woman which in turn decreases the severity of the hot flash.

Clothing items are made, in some embodiments, with characteristics similar to those of the wipes. Worn at night, pajamas made according to the present invention relieve menopausal symptoms and allow the woman to sleep with less difficulty. Worn during the day, such clothing relieves such symptoms without drawing attention to the wearer.

### DETAILED DESCRIPTION OF THE INVENTION

The following description is based on embodiments of the invention and should not be taken as limiting the invention with regard to alternative embodiments that are not explicitly described herein.

The ancients say that menopause is a time of growing wisdom, power, and knowledge in a woman's life. Menopause is not simply the end of a woman's childbearing years, it is the beginning of a new stage. During this time, women change on a cellular level, a true metamorphosis. Like puberty and pregnancy, menopause is packed with physical and psychological changes caused by shifts in a woman's hormonal makeup.

Although the transition through menopause can be gradual and smooth, many women experience much difficulty. Because of this, and even though menopause is a natural progression in life, many women face it with trepidation. Hot flashes, night-time sweats, changes in libido, and an increased risk of developing osteoporosis, cardiovascular disease, and breast cancer are just a few of the concerns of women entering this stage of life.

Over 60 million women in the United States reach menopause in a given year. Some women start this change in their middle to late thirties, but the majority of women usually begin during their forties or fifties. Peri-menopause can begin as early as the late thirties. (Peri-menopause is defined as the two to eight years preceding menopause and the year after menopause.) Peri-menopause is a time of hormone fluctuations that may begin fairly subtly, then slowly become more exaggerated as a woman approaches menopause. Women also usually have their first hot flashes and night sweats during this time. An individualized process, no woman experiences menopause in exactly the same way. Menopause symptoms range from none to many and last for varying lengths of time up to a period of five years. For most women, the most intense period of menopause lasts for two to five years. During this time, hot flashes and night sweats are often frequent and intense.

Many women say that hot flashes are the most troublesome side effect of menopause. Hot flashes happen when blood vessels erratically dilate and constrict. Blood flow increases, body temperature rises, and the heart pumps faster. Sweating usually accompanies hot flashes.

After this intense period of hot flashes and night sweats, women proceed into post-menopause, which lasts the rest of their lives.

Cultural background, diet, lifestyle, genetics, and knowledge of the menopause process play key roles in how a woman experiences menopause. Supporting her body with good choices helps keep a woman in balance, and is a key to helping her get through this intense time.

Traditionally, women have been offered a very limited range of treatment options to help their bodies adjust to declining estrogen levels and other physical changes. Hormone Replacement Therapy (“HRT”) is a popular treatment course, even though approximately 80% of women do not need any kind of HRT. The decision to use HRT is, however, a difficult one, because of the growing concern of HRT's side effects in both the short and the long term. Many women, therefore, prefer to have more natural health care options. As menopause is a natural stage of life, rather than a disease, natural remedies should be appropriate.

Aromatherapy is one of the natural health care options shown to provide relief to menopause symptoms. Aromatherapy is an ancient art using the essential oils of plants in medicine, spicery, and perfumery. Formed in petals, leaves, roots, resin, barks, stems, seeds, grasses, stalks, and rinds, an essential oil is generally the part of the plant that provides the plant's aroma. Depending on the plant, essential oils can be stored in special resin cells, glandular hairs, pockets and reservoirs, or even in the spaces between cells. For a plant, the essential oil is just that: essential. These oils have traditionally been associated with the spiritual quality of life's energy, so to use the essential oil of a plant is to use the very best of what a plant has to offer.

Aromatherapy has appeared in recipes since the beginning of recorded history, especially in reference to healing and cleansing. From the Egyptians, to the Babylonians and Arabs, to the ancient Greeks, to the Romans following the precepts of Hippocrates, essential oils were used in the bath for health purposes: “The way to health is to have an aromatic bath and a scented massage everyday.”

As the modern age of the 19th century progressed, aromatherapy and the use of essential oils were regarded as quaint stories of past cultures. Not until a cosmetic chemist of the early 1900s began scientific research did their use revive. René-Maurice Gattefossé devoted his life to researching the healing properties of essential oils after a providential accident in his perfume lab. After burning his arm badly he thrust it into the nearest cold liquid, which happened to be a vat of lavender oil. The pain decreased substantially and, rather than becoming a normal bum site of inflammation and blistering, the injury healed quickly without a scar.

Essential oils treat the whole person to make menopause a more positive experience. Specifically, these natural oils can be used to ease menopause symptoms, especially hot flashes. They also work to balance hormone levels, restore confidence, and comfort the emotions.

Essential oils are most effective when taken by inhalation or by absorption through the skin into the body. They are least effective therapeutically when taken orally. They are very efficient in leaving the body through exhalation and perspiration which means they are generally non-toxic.

Peppermint, lavender, and eucalyptus oils work best on menopause symptoms, including hot flashes. By applying these oils to a wipe, a woman benefits in two ways: from inhaling the aroma and from absorption of the oil through her skin.

Peppermint oil has a calming and numbing effect on menopause symptoms such as anxiety, feeling ill at ease, headaches, feelings of dread, apprehension, doom, and depression, all of which can bring on hot flashes. Peppermint oil when applied topically also has a soothing and cooling effect on the skin which is very effective in reducing the “over-heated” feeling associated with hot flashes.

Eucalyptus and lavender oils provide relief for such menopause symptoms as itchy, crawling skin, dizziness, mood swings, irritability, disorientation, and mental confusion, all of which can induce hot flashes.

The following five cases studies show how the effects of essential oils vary among women. 100% pure essential oils of lavender, peppermint, and eucalyptus were used. Each test subject was given three pads for each of the three different oils. Five drops of each oil were added to a pad. All test subjects are, of course, female.

The first test subject is a black woman in menopause. She found the smell of lavender oil “very calming and [it] relieved my hot flash.” Peppermint oil was “very cool on the body; I did not get my usual hot flashes for one hour.” About eucalyptus oil, she said: “I felt very relaxed, no anxiety with my hot flashes.”

The second test subject is another black woman in menopause. She “applied [lavender oil] during hot flash and felt immediate relief.” On peppermint oil, she said: “[I] applied before my usual hot flash, and I did not get it, this is great!” For her, eucalyptus oil “smells wonderful. I felt very calm.”

The third test subject is a white woman in peri-menopause. She found that lavender oil “works best during hot flash. I felt a lot better.” About peppermint oil, she said: “This was the best, felt very cool and refreshing, my hot flash ended, the effects lasted for about ten minutes!” On eucalyptus oil, she said, “I felt less anxious after applying during my hot flash, my head felt clearer. I had more energy.”

The fourth test subject is an Asian woman in peri-menopause. She said about lavender oil: “I used it as soon as I received the cotton pads with the oils. That was in the morning and I did not get a hot flash until after lunch. I was amazed. I usually get a couple before lunch!” She found peppermint oil to be “just as effective but felt really cool and refreshing. I did not feel as hot and sweaty as I usually do.” On eucalyptus oil, she said: “I rubbed this on my pulse points: neck, armpits, between my breasts. It felt good and smelled good. I felt very calm and relaxed. I had fewer hot flashes.”

The fifth test subject is an Hispanic woman in menopause. About lavender oil, she said: “I felt some relief, I just touched the pad with the oil around my hair line and I felt immediately more relaxed.” On peppermint oil, she said: “This worked best. It felt very cool. I tried applying it when I was not ‘flashing,’ and it seemed to keep my hot flashes away. When applied during the hot flash, I felt cooler and calmer immediately.” On eucalyptus oil, she said: “This helped overall, I felt like my head was clearer, I felt relaxed and not worried about having a hot flash for once. I liked this the best!”

Based on these and other experiences, the present invention provides cooling clothing and wipes to alleviate some of the symptoms of menopause, especially hot flashes and night sweats.

A wipe is made of a wicking material that does not disintegrate or leave a residue as do current tissues. Examples of appropriate material include woven cloth, non-woven cloth, polyester, and nylon. The wipe can carry an essential oil and a cooling agent, such as menthol or eucalyptus oil. When drawn across the skin, the wipe draws perspiration away from the skin while providing a cooling effect. The cooling agent is compatible with make-up. The wipe may be disposable or reusable.

The wipe can also carry other compounds to, for example, remove excess skin oil. A soothing agent, such as black cohosh, vitex, or dong quai, can be added to relax the woman which in turn decreases the severity of the hot flash.

Clothing items are made with characteristics similar to those of the wipes. Worn at night, pajamas made according to the present invention relieve menopausal symptoms and allow the woman to sleep with less difficulty. Worn during the day, such clothing relieves such symptoms without drawing attention to the wearer.

In view of the many possible embodiments to which the principles of the present invention may be applied, it should be recognized that the embodiments described herein with respect to the drawing figures are meant to be illustrative only and should not be taken as limiting the scope of the invention. Those of skill in the art will recognize that some implementation details are determined by specific situations. Therefore, the invention as described herein contemplates all such embodiments as may come within the scope of the following claims and equivalents thereof.

We claim:

1. A wipe for easing symptoms of menopause, the wipe comprising:
a wicking fabric; and
a cooling agent carried by the wicking fabric.

2. The wipe of claim 1 wherein the wipe is compatible with makeup.

3. The wipe of claim 1 wherein the wicking fabric is selected from the group consisting of: woven cloth, non-woven cloth, polyester, and nylon.

4. The wipe of claim 1 wherein the cooling agent is selected from the group consisting of: menthol and peppermint oil.

5. The wipe of claim 1 further comprising:
a calming agent selected from the group consisting of: eucalyptus oil and lavender oil.

6. The wipe of claim 1 further comprising:
a soothing agent carried by the wicking fabric.

7. The wipe of claim 6 wherein the soothing agent is selected from the group consisting of: black cohosh, vitex, and dong quai.

8. The wipe of claim 6 wherein the cooling agent and the soothing agent are compatible with makeup.

9. The wipe of claim 1 further comprising an element selected from the group consisting of: perfume, skin conditioner, moisturizer, sunscreen, antibiotic, surfactant, vitamin, and alcohol.

10. An article of clothing for easing symptoms of menopause, the clothing article comprising:
a wicking fabric; and
a cooling agent carried by the wicking fabric.

11. The clothing article of claim 10 wherein the clothing article is compatible with makeup.

12. The clothing article of claim 10 wherein the wicking fabric is selected from the group consisting of: woven cloth, non-woven cloth, polyester, and nylon.

13. The clothing article of claim 10 wherein the cooling agent is selected from the group consisting of: menthol, peppermint oil, and eucalyptus oil.

14. The clothing article of claim 10 further comprising:
a calming agent selected from the group consisting of: eucalyptus oil and lavender oil.

15. The clothing article of claim 10 further comprising:
a soothing agent carried by the wicking fabric.

16. The clothing article of claim 15 wherein the soothing agent is selected from the group consisting of: black cohosh, vitex, and dong quai.

17. The clothing article of claim 15 wherein the cooling agent and the soothing agent are compatible with makeup.

18. The clothing article of claim 10 further comprising an element selected from the group consisting of: perfume, skin conditioner, moisturizer, sunscreen, antibiotic, surfactant, vitamin, and alcohol.

19. The clothing article of claim 10 wherein the clothing article is selected from the group consisting of: pajamas, tank top, shorts, pants, bra, and panties.
